(19) United States
(12) Patent Application Publication
(10) Pub. No.: US 2009/0193878 A1
(43) Pub. Date: Aug. 6, 2009

(54) METHOD FOR DETECTING LEVELS OF OVERALL VISCOSITY OF A SAMPLE OF WHOLE BLOOD

(75) Inventors: Alfredo Ciotti, Udine (IT); Paolo Galiano, Padova (IT)

Correspondence Address: HAMRE, SCHUMANN, MUELLER & LARSON, P.C., P.O. BOX 2902, MINNEAPOLIS, MN, 55402-0902, US

(73) Assignee: ALIFAX HOLDING SPA, Polverara (PD) (IT)

(21) Appl. No.: 12/359,501

(22) Filed: Jan. 26, 2009

Related U.S. Application Data
(63) Continuation of application No. PCT/EP2007/057531, filed on Jul. 20, 2007.

(30) Foreign Application Priority Data
Jul. 24, 2006 (IT) UD2006A000184

(51) Int. Cl. G01N 11/04; G01N 33/49

(52) U.S. Cl. 73/54.04; 356/39

(57) ABSTRACT

A method for detecting levels of viscosity of a sample of whole blood, comprising the steps of providing a curve of the kinetics of optical density of the sample of whole blood, said curve comprising a first point (b) having a value of optical density and a point (c) of minimum value of said optical density; determining the value of the drop between the value of optical density in correspondence with the point (b) and the minimum value of optical density in correspondence with the point (c); obtaining information, based on the value of drop, on the value of viscosity of said sample of whole blood, wherein a low value of the drop of optical density corresponds to a higher level of viscosity of the sample of blood and a high value of the drop corresponds to a lower level of viscosity of the sample of blood.

### FIELD OF THE INVENTION

The present invention concerns a method for detecting levels of high overall or intrinsic viscosity of a sample of whole blood.

In particular, the method for detecting the viscosity according to the present invention is based on determining and correlating the entity of the drop in optical density that is deduced from the relative curve, known as syllectogram, of the sample of whole blood when the latter is subjected to measurement of the erythrocyte sedimentation rate (ESR).

### BACKGROUND OF THE INVENTION

There is a growing interest, on the part of clinicians, in blood rheology, and in particular in the study of the aggregation of the red corpuscles, which deals with the characteristics of the blood flow. Indeed, it has been widely demonstrated that the blood flow does not depend only on the action of the cardiac pump and on the resistance imposed by the vascular geometry, but also on the resistance offered by the blood itself. In the most varied pathological conditions, the alteration of the blood flow is an expression not only of structural or functional alterations of the heart and blood vessels, but also of the interaction of these with the characteristics of the blood proper.

Consequently, the treatment of alterations of the blood flow involves considerations concerning not only the heart and vessels, but also the blood; modifying its rheological properties, that is, its overall or intrinsic viscosity, can constitute a useful evolution of current therapeutic strategies.

Plasma viscosity is an indicator of an acute and chronic illness and, together with fibrinogen and the white cell count, is considered a factor of risk for ischemic heart disease. Possible causes of high plasma viscosity are the high presence of fibrinogen and plasma proteins, especially the very large ones which form bridges between red corpuscles and determine the phenomenon of erythrocyte aggregation in rouleaux.

In fact, many clinical studies confirm the statistically significant association between high levels of fibrinogen and arterial thrombotic illnesses, the incidence of which increases in proportion to age and which represent one of the main causes of death.

Fibrinogen is a protein with a high molecular weight which, apart from being the precursor of fibrin in the coagulation cascade, is the factor which most affects plasma viscosity. Furthermore, fibrinogen is an asymmetrical molecule present in the blood, with normal concentration values in the blood comprised between 200-400 mg/dl. This asymmetry implies that, even when there is a very low fibrinogen percentage, its effect on the overall intrinsic viscosity is in any case considerable.

Viscosity, that is, the intrinsic resistance opposed by the fluid to flow, can be defined as the ratio between the force needed to keep the threads of the fluid in reciprocal movement, and the gradient of speed between contiguous threads.

We can imagine that the blood flow, in rectilinear vascular segments, consists of the movement in several parallel layers of fluid, each having a different speed: the speed of the individual layers increases progressively as they gradually move away from the wall of the vessel towards the center.

In the blood the speed depends substantially on the number of red corpuscles, the concentrations of plasma proteins, in particular fibrinogen, and the erythrocyte deformability.

The resistance opposed to movement, however, increases with low speed values, whereas it decreases when the speed is increased: this behavior depends on the ability of the erythrocytes to aggregate and deform.

The first phenomenon consists in the capacity of the red corpuscles to form three-dimensional aggregates able to decrease the blood fluidity.

The plasma protein macromolecules are responsible for aggregation, in particular fibrinogen: said molecules neutralize the negative surface charges that promote the reciprocal repulsion of the erythrocytes.

With the increase in the flow speed, the aggregates are dispersed, whereas the erythrocytes and macromolecules tend to dispose themselves in the main direction of the vessel. The red corpuscles deform in an ellipsoidal direction, with a greater axis parallel to that of the vessel.

With regard to the effects on coagulation of the viscosity of the blood, different studies have shown alterations in the laboratory tests, such as reduction in the platelets, dilution of the coagulation factors or other, without an obvious increase in hemorrhage.

Therefore, in the medical and diagnostic field, there is a great need to evaluate, in a reliable, quick and economical manner, anomalous levels or states of overall or intrinsic viscosity of the blood, in order to be able to signal possible levels of potential risk for the patient, inflammatory situations in progress or various pathologies.

The Article by Johannes G. G. Dobbe, “Syllectometry: The Effect of Aggregometer of Red Blood Cell . . . ” IEEE TRANSACTIONS OF BIOMEDICAL ENGINEERING, Vol. 50, NO. 1, January 2003, discloses an analysis of the shape of the syllectogram defining a stage of shape-recovery in which the blood cells, after the sudden stop, lose collectively their alignment and then return to their biconcave shape. In particular, Dobbe et al. investigates the possibility that the geometry of the different aggregometer available on the market influence the shape of the syllectogram. To this end, Dobbe et al. proposes a new mathematical function of the tri-exponential type so as to analytically describe the syllectogram. Such a mathematical function takes into account, besides the aggregation phase of the rouleaux after the peak of the syllectogram, the recovery-shape phase of the red corpuscles that happens after a brusque interruption of the blood flow and until the peak of the syllectogram has been reached, that is, before the aggregation phase of the rouleaux. However, this document does not teach how to obtain information from the syllectogram about the viscosity of the blood plasma.

U.S. Pat. No. 4,352,557 and EP-A2-239.690 disclose methods for measuring the aggregation rate of blood cells by optical measurements.

Purpose of the present invention is therefore to perfect a method which allows to detect levels of anomalous viscosity of the blood and therefore to identify possible states of potential risk for the patient, inflammatory situations in progress or various pathologies, in a reliable, quick and economical manner.

The Applicant has devised, tested and embodied the present invention to overcome the shortcomings of the state of the art and to obtain these and other purposes and advantages.

### SUMMARY OF THE INVENTION

The present invention is set forth and characterized in the independent claim, while the dependent claims describe other characteristics of the invention or variants to the main inventive idea.

In accordance with the above purpose, a method for detecting levels or states of overall or intrinsic viscosity, in particular anomalous viscosity, of a sample of whole blood comprises a preliminary step of constructing a curve of the kinetics of the optical density, or syllectogram, of the sample of whole blood, in which the curve comprises a first point having a determinate value of optical density and a point of minimum value of optical density. Once the curve has been obtained, the invention provides to measure the difference, or in any case to draw indications on its entity, between the value of optical density of the first point of the syllectogram, corresponding to the moment of the syllectogram, wherein the aggregations of the red corpuscles have been eliminated, absence of rouleaux, by means of the flow along the capillary, and the new value, minimum point of the optical density, in this case absorbance, detected following the sudden stoppage of the flow along the capillary, the stopped flow, corresponding to the point of the syllectogram which describes the start of the aggregation kinetics.

Depending on the entity of this difference, according to the invention, it is possible to draw indications on the overall viscosity of the sample of whole blood.

In particular, Applicant has surprisingly found a correlation between the entity of the drop in optical density identifiable in the syllectogram and the intrinsic viscosity of the blood, establishing that a low value of the drop in optical density corresponds to a high concentration of proteins, in particular fibrinogen and triglycerides, which is the cause of a higher level of viscosity, whereas a high value of the drop in optical density corresponds to a decreased concentration of proteins, particularly fibrinogen and triglycerides, cause of a lower level of intrinsic viscosity in the whole blood subjected to examination.

Therefore, the present invention allows to process a factor which, with a good approximation, allows to identify high, low and medium levels of intrinsic viscosity in the sample of whole blood, and therefore to identify states of potential risk for the patient, inflammatory situations in progress or various pathologies, in a reliable, quick and economical manner.

Advantageously, the invention can be applied to any device or system able to cause kinetics of optical density describable with a diagram known as a syllectogram.

The invention is also easy to apply, since the sample of whole blood to be analyzed does not require a pre-analytical preparation step, such as centrifugation, sedimentation or other.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a diagram representing the syllectogram of a sample of whole blood, where the optical density is shown on the y-axis and the time in seconds on the x-axis; and

FIG. 2 is a statistical distribution of the number of classes of photometric drop for a plurality of blood samples analyzed by means of the present invention.

### DETAILED DESCRIPTION OF A PREFERENTIAL FORM OF EMBODIMENT

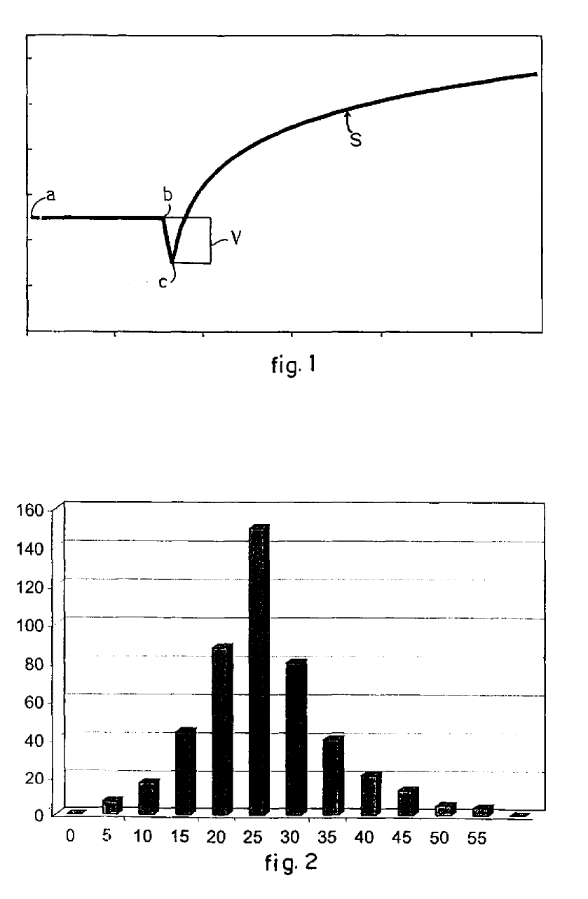

According to the present invention, FIG. 1 shows a curve S of the kinetics of optical density, or syllectogram, of a sample of whole blood to achieve a method for detecting, measuring and predicting levels of intrinsic or overall viscosity, in particular anomalous viscosity, of the sample of whole blood, that is, high, low and medium levels.

To construct the curve S, in a known manner, the sample of whole blood is disposed in a containing seating associated with an optical detection device, comprising at least a light emitter and a mating light receiver, the rouleaux which have formed are made to break up by means of an interruption of the quiet state, or uniform motion, of the sample of whole blood. The kinetics is detected by the optical detection device which constructs the curve S by means of a suitable algorithm, for example using the method and apparatus described in the European patent application EP-A.1.098.188 in the name of the Applicant.

The curve S, or syllectogram, describes a flow step, at constant speed, of the sample of whole blood, for example through a capillary constituting a reading cell, between the points “a” and “b” of the curve S, in which there is a break-up of the rouleaux with a substantially constant optical density. In this step the sample of whole blood is made to flow, for example along the capillary, at the speed of 200 mm/s, and the red corpuscles deform and tend to occupy a position of balance, near the central axis of the capillary. In this situation a greater quantity of light reaches the photometric sensor.

The greater the quantity of proteins present, such as fibrinogen, and triglycerides, and hence the greater the viscosity of the liquid transporting the red corpuscles, or blood plasma, the greater the difficulty for the individual red corpuscle to reach the axis of balance, substantially at the center of the capillary. Vice versa, the smaller the quantity of proteins present, such as fibrinogen, and triglycerides, and hence the lesser the viscosity of the transport liquid, the smaller the obstacle will be for the red corpuscles in reaching the axis of balance. In the present description, with the expression “viscosity” or “viscosity of blood” we mean the viscosity of the liquid transporting the red corpuscles, or blood plasma, in a sample of whole blood.

The point “b” corresponds to the sudden stopping point of the flow, or stopped flow, through the capillary, as described in EP'188.

By brusquely stopping the flow through the capillary, the red corpuscles, which by and large have accumulated at the center of the capillary during the flow in the capillary at constant speed, tend to distribute themselves homogeneously in the volume of the surrounding transport liquid. This effect is recorded and made explicit by the syllectogram with an instantaneous reduction of the light reaching the sensor of the photometer, from point “b” to point “c”.

Point “c”, which is the minimum point of the curve S, also represents the start of the kinetics of aggregation of the red corpuscles and the formation of the rouleaux.

Point “c” corresponds to the moment of the kinetics of optical density wherein the latter is most correlated with the hematocrit and hemoglobin value of the blood samples. The integer of the area subtended to the curve S, which has point “c” of the syllectogram as its base line, is indicated as M-index in literature.

The method according to the invention provides to identify the entity of the “V-INDEX” difference or drop in the optical density, indicated by V in FIG. 1, between the value of optical density of the point “b” where the kinetics starts, and the value of optical density at point “c”, minimum point of the curve S.

In substance, the value of the “V-INDEX” difference is the drop, or amplitude of the drop, which the curve S makes between point “b” and point “c”, that is, an absolute value of optical density of the sample of whole blood, measured in this case according to the method described in EP'188, which uses an optical path of the capillary associated with the 0.8 mm photometer.

According to the value of the “V-INDEX” difference, repeatable and different for every sample analyzed, it is possible to draw indications, with a good approximation, on the value of overall viscosity, or viscosity factor, of the sample of whole blood (primary). The invention provides to repeat the measurement of the drop in optical density “V-INDEX” for a plurality of different blood samples, and thus to determine a distribution in frequency of a plurality of classes of photometric drop and to associate with each class of photometric drop a correlated approximate value, for example high, medium, low, of intrinsic viscosity.

FIG. 2 shows the distribution in frequency of the classes of photometric drop found by experimentation according to the invention as carried out by Applicant on 469 different blood samples. On the x-axis the classes of photometric drop are shown, identified by the absolute value of drop in optical density, equal to the above-mentioned “V-INDEX”. This value is shown on the x-axis deliberately multiplied by 1000, for a clearer exposition, that is, with a linear scale value using thousandths of percentage absorbance.

Therefore, for example, where 50 is indicated, this means a drop in optical density corresponding to about 0.05 units of absorbance, whereas where 10 is indicated, this means a drop in optical density corresponding to about 0.01 units of absorbance and so on.

Again in FIG. 2, the y-axis shows the number of blood samples belonging to each class of photometric drop.

The average value of the samples analyzed is 23.8 while the value of the median, which represents the typical value of the series of data, is 24.0. Moreover, the equality of the average and median values ensures a balanced distribution of the values.

According to the invention, in the identification step, the value of the “V-INDEX” difference is assigned to or associated with one of the pre-determined classes of photometric drop.

In particular, an assignment of the value of the “V-INDEX” difference to a class of photometric drop with a low value is an indicator of high overall viscosity of the sample of whole blood, and hence possible pathological risks for the patient.

Furthermore, according to the invention, said low value class of photometric drop, or high value of viscosity, is an indicator, with a good approximation, of a high concentration of fibrinogen and/or triglycerides in the sample of whole blood.

From the experimentation carried out, it is clear that the low value class of photometric drop can be quantified as comprised in a range between about 0 and 10 of class of photometric drop, as shown in FIG. 2, that is, between about 0 and 0.01 absolute value of optical density, in this case expressed in units of absorbance.

In particular, from the experimentation carried out, it emerges that 24 of the 469 samples analyzed, corresponding to about 5% of the total, have a drop in optical density lower than or equal to 10, as indicated in FIG. 2.

According to the invention, an assignation of the value of the “V-INDEX” difference to a class of photometric drop with a high value, for example higher than 40 (FIG. 2), is an indicator of low overall viscosity of the sample of whole blood.

From in-depth clinical tests made on these 24 samples the presence of high fibrinogen emerged in 17 cases and high triglycerides in 4 cases, which, as is known, increase the overall viscosity of the blood.

Therefore, the “V-INDEX” value found from the drop of optical density quantified and divided into classes of photometric drop, according to the invention, is an alarm indicator for blood samples estimated empirically with a high overall viscosity and allows to alert the analyst and the clinician quickly, so that an in-depth diagnostic investigation may be made.

It is clear that modifications and/or additions of steps may be made to the method for detecting levels of overall viscosity of a sample of whole blood as described heretofore, without departing from the scope of the present invention.

1. A method for detecting levels of overall or intrinsic viscosity of a sample of whole blood, comprising the steps of:
i) disposing the sample of whole blood in a containing seating associated with an optical detection device, comprising at least a light emitter and a mating light receiver, the sample of whole blood being made to flow at constant speed through a capillary constituting a reading cell, wherein the red corpuscles deform and tend to occupy a position of balance, near the central axis of the capillary, the rouleaux which have formed being made to break up by means of an interruption of the quiet state, or uniform motion, of the sample of whole blood so that the red corpuscles, which by and large have accumulated at the center of the capillary during the flow in the capillary at constant speed, tend to distribute themselves homogeneously in the volume of the surrounding transport liquid, or blood plasma, the kinetics being detected by said optical detection device which constructs a curve (S) of the kinetics of optical density, or syllectogram, of said sample of whole blood, in which said curve (S) comprises a first point (b) having a determinate value of optical density and that corresponds to the sudden stopping point of the flow, or stopped flow, through the capillary, and a point (c) of minimum value of said optical density;
ii) determining the value or entity of the difference (V) or drop between the value of optical density in correspondence with said point (b) and the minimum value of optical density in correspondence with said point (c);
iii) obtaining information, based on said value of difference (V) or drop, on the value of overall viscosity of the liquid transporting the red corpuscles, or blood plasma of said sample of whole blood, wherein the greater the quantity of proteins present, such as fibrinogen, and triglycerides, and hence the greater the viscosity of the liquid transporting the red corpuscles, or blood plasma, the greater the difficulty for the individual red corpuscle to reach the axis of balance, substantially at the center of the capillary, vice versa, the smaller the quantity of proteins present, such as fibrinogen, and triglycerides, and hence the lesser the viscosity of the transport liquid, the smaller the obstacle will be for the red corpuscles in reaching the axis of balance, so that a low value of said drop of optical density corresponds to a higher level of viscosity of the plasma of the sample of blood and a high value of said drop corresponds to a lower level of viscosity of the plasma of the sample of blood.

2. The method as in claim 1, wherein said steps i) and ii) are repeated a plurality of times, for a plurality of different blood samples, in order to determine a distribution in frequency of a plurality of classes of photometric drop and to associate with each class of photometric drop a value bases on said difference (V) between the values of optical density.

3. The method as in claim 2, wherein said value defining a class of photometric drop is an absolute value of optical density of said sample of whole blood.

4. The method as in claim 2, wherein said step of obtaining information on viscosity provides to associate said difference (V) with one of said classes of photometric drop.

5. The method as in claim 3, wherein an assignation of said value of difference (V) to a class of photometric drop with a low value is an indicator of a probable high overall viscosity of said sample of whole blood, and vice versa.

6. The method as in claim 5, wherein said low value of said class of photometric drop is an indicator of a probable high concentration of fibrinogen in said sample of whole blood.

7. The method as in claim 5, wherein said low value of said class of photometric drop is an indicator of a probable high concentration of triglycerides in said sample of whole blood.

8. The method as in claim 5, wherein said low value of said class of photometric drop is comprised between about 0 and 10.

9. The method as in claim 8, wherein said low value of said class of photometric drop comprised between about 0 and 10 corresponds to an absolute value of optical density, expressed in units of absorbance, comprised between 0 and 0.01.
